(19) United States
(12) Patent Application Publication
(10) Pub. No.: US 2019/0015799 A1
(43) Pub. Date: Jan. 17, 2019

(54) A Rigid Housing for Holding a Flexible Bag

(71) Applicant: GE HEALTHCARE BIO-SCIENCES AB, UPPSALA (SE)

(72) Inventors: Klaus Gebauer, Uppsala (SE); Peter Toreheim, Uppsala (SE); Patrik Akerstrom, Uppsala (SE); Patrick Jonsson, Uppsala (SE)

(21) Appl. No.: 16/066,209

(22) PCT Filed: Jan. 4, 2017
(86) PCT No.: PCT/EP2017/050103

§ 371 Date: Jun. 26, 2018

(30) Foreign Application Priority Data
Jan. 8, 2016 (GB) 1600319.6

(51) Int. Cl. B01F 13/00; C12M 1/00; B01F 15/00; C12M 3/00

(52) U.S. Cl. CPC B01F 13/00; C12M 23/14; C12M 23/48; C12M 23/26; B01F 15/0085

(57) ABSTRACT

A rigid housing comprising a bottom part and at least one wall part, said bottom part and said at least one wall part together defining an internal volume when the bottom part is provided below the at least one wall part in a processing position, said rigid housing being arranged for holding a flexible bag within the internal volume, wherein the bottom part is rotatable about an axis of rotation, wherein said axis of rotation is substantially parallel to a longitudinal axis of said rigid housing such that the bottom part can be provided in a loading position in which the bottom part has been rotated out from the position below the at least one wall part.

### TECHNICAL FIELD OF THE INVENTION

The present invention relates to a rigid housing for holding a flexible bag and to a method for loading a flexible bag into a rigid housing. The flexible bag can be a single use bioreactor.

### BACKGROUND OF THE INVENTION

Flexible bioreactor bags can be provided inside a rigid housing. Different solutions have been described for loading the flexible bag into the rigid housing.

A standard solution for loading the flexible bag into the rigid housing is to utilize an opening in the reactor wall to insert the collapsed bag through this opening (XDR Bioreactor, GE Healthcare). A reinforcement plate is then used to support the bag across the surface of the opening during processing and when filled with liquid. This loading method is applicable to bags that can be collapsed to a small size. Another method of loading a flexible bag is to utilize one or multiple door segments in the rigid housing of the bioreactor. By closing the door(s) after bag loading, the rigid housing does support the bag during processing and when filled with liquid. The flexible bag may also be loaded through an opening at the top of the rigid housing. However this method is typically only applicable for smaller bioreactors with a height of the rigid housing not exceeding approximately 50 cm.

The above described bag loading methods all have the disadvantage of the operator needing to access the internal of the bioreactor and the rigid housing to arrange the bag in its required position, for example by docking a magnetic impeller in the bag to a magnetic drive plate in the bottom of the rigid housing. This issue with poor usability and ergonomics is in proportion to the size of the reactor.

Another loading method that provides better access to the bottom of the rigid housing is described in the product Mobius® 2000 Liter Single-Use Bioreactor from Millipore. Here a bottom loading drawer is used. The drawer is guided on trails and can be drawn out below the rigid housing. A single-use bioreactor can be provided inside the drawer which then is pushed back to a position below the rest of the rigid housing. Another example can be seen in the ABEC CSR-Bioreactor™. Here a small carriage is provided as a bottom part of the rigid housing. The carriage can be moved to a loading position outside the rigid housing. The single use bioreactor is provided on the carriage which then is moved back into the rigid housing.

A drawback with the movable bottom part of the Millipore device is that cable and/or tubing carriers have to be employed to accommodate the change in distances between cable and/or tubing connection points at the bioreactor bottom and the system, respectively. Due to the linear motion and displacement of the movable bottom, these cable carriers are not static but need to be movable and flexible, which requires additional space underneath the rigid housing and bioreactor.

While the ABEC device mentioned above does not necessarily need a flexible cable carrier to accommodate a displacement of tubing and/or electric cables, it requires a physical connection and disconnection of tubing and cables in between the carriage and the system to allow for a removal of the carriage in the first place.

### SUMMARY

An object of the present invention is to provide a rigid housing arranged for holding a flexible bag where the flexible bag can be loaded into the rigid housing in an easy way with good ergonomics for the operator.

A further object to the invention is to provide a method for easy loading of a flexible bag into a rigid housing.

This is achieved in a rigid housing comprising a bottom part and at least one wall part, said bottom part and said at least one wall part together defining an internal volume when the bottom part is provided below the at least one wall part in a processing position, said rigid housing being arranged for holding a flexible bag within the internal volume, wherein the bottom part is rotatable about an axis of rotation, wherein said axis of rotation is substantially parallel to a longitudinal axis of said rigid housing such that the bottom part can be provided in a loading position in which the bottom part has been rotated out from the position below the at least one wall part.

This is also achieved in a method for providing a flexible bag into a rigid housing as described above, said method comprising the steps of:
rotating the bottom part of the rigid housing to a loading position;
loading the flexible bag into the bottom part;
rotating the bottom part back to a processing position where the bottom part and the at least one wall part forms a rigid housing with an internal volume.

This is also achieved in a rigid housing comprising a bottom part and at least one wall part comprising a front part, said bottom part and said at least one wall part together defining an internal volume when the front part is provided in a processing position, said rigid housing being arranged for holding a flexible bag within the internal volume, wherein the front part is attached to a multiple joint configuration which also is attached to another part of the rigid housing such that the front part can be provided both in a closed position, also called a processing position, where the front part together with the rest of the side wall enclose the internal volume and in a folded up position, also called a loading position, where the front part is folded up behind the rest of the side wall and access is given to the bottom part for loading of a flexible bag into the bottom part.

Hereby the bottom part or a wall part can be rotated out from the other part such that an operator gets good access to the bottom part for loading the flexible bag therein. While the loading of the flexible bag and a corresponding loading position will be discussed hereafter, it is understood that the technical and ergonomic advantages of the invention with its improved loading position during bag loading equally apply during the removal of the bag. Furthermore with this invention there is no need for specifically designed arrangements, such as flexible carriers, for accommodating a movement and displacement of tubing and/or electrical cables and connections underneath the bioreactor bottom. Instead, tubing and/or electrical cables can be routed along the rotating parts as long as they allow sufficient bending along the points of rotation to follow a change in angular alignment of parts and/or guide means such as for example rotating arms and holders. Furthermore the space below the bottom part will be free (no wheels or trails as in some of the previous used methods) in both loading and processing positions which will allow for better access for service and maintenance. Said tubing routed to the bioreactor bottom may include tubing for heat exchanger fluids employed for heating or cooling in case that the bioreactor bottom is designed with a double jacket to accommodate heat exchange features and transfer heat to or from the flexible bag and bioreactor fluid volume to the jacketed vessel or vice versa. Said electrical connections and cables routed to the bioreactor bottom may include wiring to heat blankets covering parts of the bioreactor bottom to accommodate for heat exchange features to transfer heat from the bioreactor bottom to the flexible bag and the bioreactor fluid. The above mentioned tubing of wiring for heat exchanger features are preferably attached fixed and permanently to the bioreactor bottom as they are re-used and without need for replacement and re-connection in between processing runs, in contrast to the single-use bag. Another example for tubing routed to the bioreactor bottom is tubing for gas transfer to the bioreactor bag. As the bioreactor typically comes equipped with a sterilizing grade inlet filter and a connection for inlet gas, the tubing and connector means for connecting the gas inlet tubing to the single-use bioreactor can be routed permanently to the bioreactor bottom and the connection point to the single-use bioreactor bag.

Embodiments of the invention are described in the dependent claims and in the following detailed description.

### BRIEF DESCRIPTION OF THE DRAWINGS

FIG. 1a shows schematically a rigid housing according to one embodiment of the invention.

FIG. 1b shows schematically a rigid housing according to another embodiment of the invention.

FIG. 1c shows schematically a rigid housing according to another embodiment of the invention.

FIG. 1d shows schematically a rigid housing according to another embodiment of the invention.

FIG. 1e shows schematically a rigid housing according to another embodiment of the invention.

FIG. 2a shows schematically a rigid housing according to one embodiment of the invention.

FIG. 2b shows schematically the rigid housing of FIG. 2a in a top view.

FIG. 3a shows schematically a rigid housing according to one embodiment of the invention.

FIG. 3b shows schematically the rigid housing of FIG. 3a in a top view.

FIG. 4a shows schematically a rigid housing according to another embodiment of the invention in a first position both in a perspective view and in a top view.

FIG. 4b shows the embodiment of FIG. 4a but in a second position.

FIG. 4c shows the embodiment of FIG. 4a in a third position.

FIG. 5 is a flow chart of a method according to the invention.

### DETAILED DESCRIPTION OF THE EMBODIMENTS

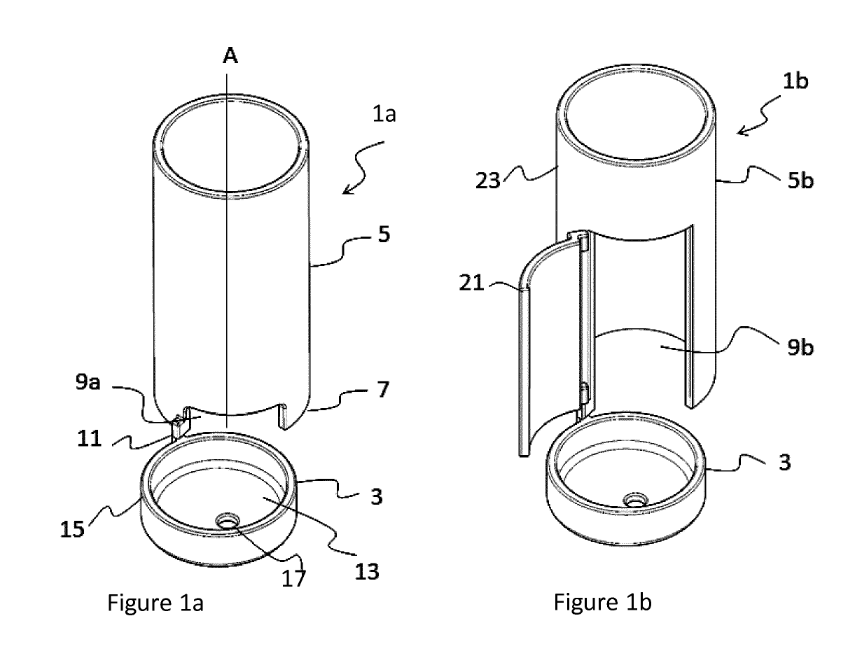

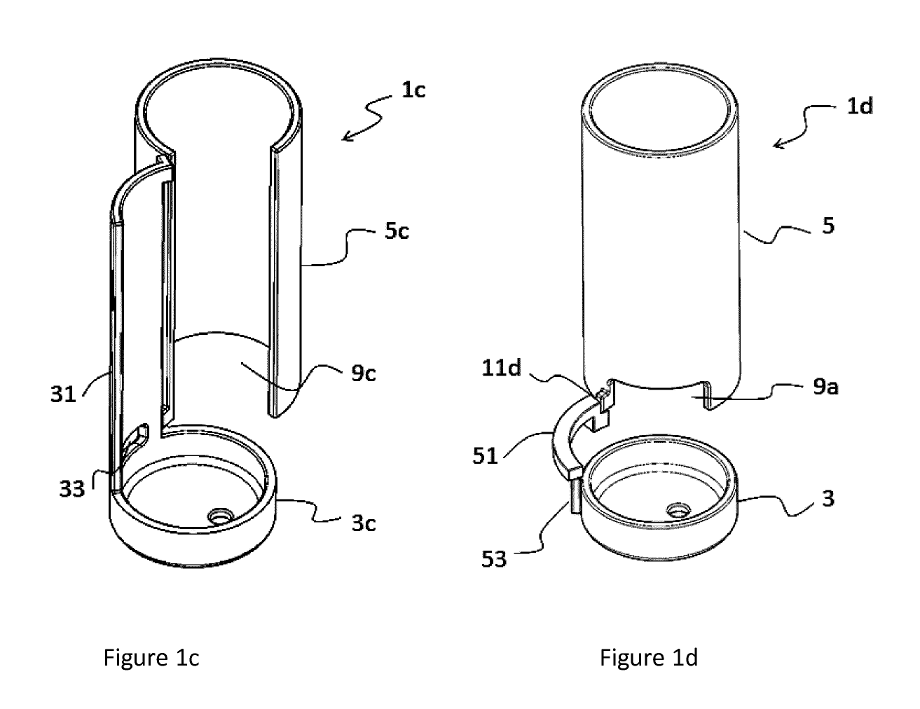

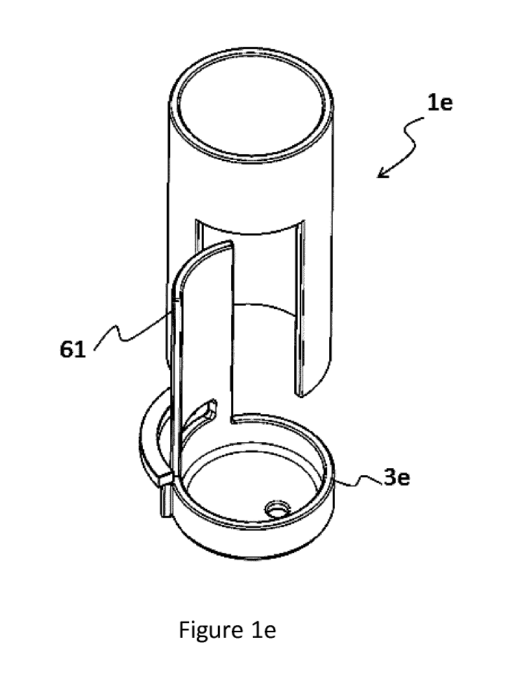

FIG. 1a-1e shows schematically different embodiments of a rigid housing according to the invention. According to one embodiment of the invention a rigid housing is provided which comprises a bottom part and at least one wall part. The bottom part and the at least one wall part define together an internal volume when the bottom part is provided below the at least one wall part in a processing position. The rigid housing is arranged for holding a flexible bag within the internal volume. Further according to the invention the bottom part is rotatable about an axis of rotation, wherein said axis of rotation is substantially parallel to a longitudinal axis of said rigid housing. Hereby the bottom part can be provided in a loading position in which the bottom part has been rotated out from the position below the at least one wall part. The flexible bag can be a single use bioreactor. The axis of rotation around which the bottom part is rotatable can be positioned at the wall part or outside the wall part.

In all the embodiments shown in FIGS. 1a-1e the wall part is shown to be a tubular wall however the geometrical design can be varied and still be covered by this invention. For example a box shaped part of the rigid housing and flexible bag is feasible and rectangular walls may be employed for construction of the bag and rigid housing. Other shapes and geometries of surrounding wall segments and internal volumes of the flexible bag and the rigid housing are feasible as well as combinations thereof, for example rectangular, triangular, hexagonal etc.

A more detailed description of each of the embodiments shown in FIGS. 1a-1e will follow below.

FIG. 1a shows schematically a rigid housing la according to one embodiment of the invention. The rigid housing la comprises a bottom part 3 and a wall part 5. The wall part 5 is in this embodiment formed as a tubular wall 5. In a first end 7 of the tubular wall 5 an opening 9a is provided. This opening 9a can for example facilitate access to ports of a flexible bag provided into the rigid housing. This could be ports for probes and sensors or sampling ports. More than one such opening 9a can be provided in the wall 5 and the size of the opening can be varied. The bottom part 3 is connected to the wall part 5 at its first end 7. The bottom part 3 is connected to the wall part 5 through a joint 11. According to the invention this joint 11 is provided such that the bottom part 3 can be rotated about an axis of rotation which is substantially parallel to a longitudinal axis A of said rigid housing 1a. The bottom part 3 is in this embodiment container formed, i.e. comprises a bottom plate 13 and surrounding walls 15 extending from the bottom plate forming a container together with the bottom plate 13. When loading a flexible bag into the rigid housing 1a the flexible bag is provided into the container formed bottom part 3 when the bottom part 3 of the rigid housing has been rotated out and thus separated from the wall part. This will be called a loading position. The bottom part 3 comprises further in one embodiment of the invention an opening 17. This opening 17 can for example be provided for connecting an impeller of a flexible bag to a drive head of a magnetic drive unit positioned in or underneath the bottom plate. Depending on the construction of the bioreactor, an insert, a closed surface or other solutions may be found instead of an opening in the bottom part that embody this impeller connection point. Opening 17 has been selected to exemplify the advantages of the invention in regard to the rotational translocation of the bottom part vs. the linear translocation found at prior art. The opening 17 can also be used for allowing for the transfer of liquid and/or for access to measuring of parameters or properties of the fluid internal to the bioreactor. Of course more than one opening 17 can be provided in the bottom part 3. Either in the bottom plate 13 as shown in FIGS. 1a-1e or in the surrounding walls 15 of the bottom part 3. Furthermore such an opening for allowing for the transfer of liquid and/or for access to measuring of parameters or properties of the fluid internal to the bioreactor and/or coupling a mixer element internal to the bag to an external drive unit can instead or complementary be provided in the wall part 5. The opening 9a in FIG. 1a is an exemplary such opening. The opening can be provided in another position on the wall part 5 or in a door of the wall part.

FIGS. 1a-1e illustrate that the rotation of the bottom part 3 provides easy access to the opening 17. The opening 17 does not need to be provided in the center of the bottom plate 13 of the bottom part 3 but can suitably (as shown in FIGS. 1a-1e) be provided off center in a location giving optimal access to an operator when the bottom part 3 is in loading position. This is a significant advantage in relation to previous solutions (Millipore, Abec) as the operator typically needs to position the bag impeller over the magnetic drive unit. The rotational movement provides therefore an advantage as there are typically multiple connections and interface elements at the bottom part such as for example an impeller coupling and a fluid drain port. While the impeller coupling solely needs to be accessible during installation and removal of the flexible bag, the fluid drain port should be accessible during processing and therefore needs to be positioned at the front of the bioreactor bottom for ergonomic access. With the invention presented here, the magnetic impeller coupling can be positioned toward the rear of the bottom plate during processing while assuming a front end position in the bag installation position of the bottom part, loading position. This allows to position fluid connections at the bottom part, for example for fluid draining, toward the front side of the bottom plate during processing.

In the embodiments described in relation to FIGS. 1b-1e some parts are identical to the parts of the embodiment described in FIG. 1a and those parts will have the same reference numbers and will not be described in detail again.

FIG. 1b shows schematically a rigid housing 1b according to another embodiment of the invention. In this embodiment an opening 9b in the wall part 5b is extending over a larger part of the wall part 5b than in the embodiment shown in FIG. 1a. Furthermore a door 21 is provided on hinges 23 such that the opening 9b can be closed by the door 21 and the door can be opened to get access to the internal volume defined by the bottom part 3 and the wall part 5b. Such a larger opening 9b can be suitable if an elongated mixing device, such as an impeller, is provided in the flexible bag which is to be provided into the rigid housing. Some mixing devices, such as impellers would be easier to install with a larger opening as shown in this embodiment.

FIG. 1c shows schematically a rigid housing 1c according to another embodiment of the invention. In this embodiment an opening 9c in the wall part 5c is extending over the whole height of the rigid housing 1c. Furthermore the bottom part 3c comprises a door part 31 which is connected to the bottom part 3c and follows the bottom part 3c in the rotation. The door part 31 covers the opening 9c when the bottom part 3c is provided beneath the wall part 5 in a processing position, i.e. when the bottom part 3c not is rotated out to a loading position. The door part 31 comprises in this embodiment of the invention an opening 33 through which sensors and cables to be connected to the flexible bag can be provided. An advantage with this embodiment of the invention is that tubes connected to the flexible bag to be provided inside the rigid housing can be folded over the door 31 already when the flexible bag is provided in the bottom part during loading. This will facilitate the process of installing the flexible bag within the rigid housing. These tubes can for example be tubes connected and used for addition or removal of liquids or air.

FIG. 1d shows schematically a rigid housing 1d according to another embodiment of the invention. This embodiment corresponds to the embodiment shown in FIG. 1a. The wall part 5 and the opening 9a are the same and the bottom part 3 is the same. However in this embodiment an extension rod 51 is provided. The bottom part 3 is connected via a second joint 53 to one end of the extension rod 51 such that the bottom part 3 can rotate about a second axis of rotation which is also substantially parallel with the longitudinal axis A of the rigid housing. The extension rod 51 is in turn connected via a first joint 11d to the wall part 5 such that the extension rod 51 can rotate about an axis of rotation as described above. Hereby the bottom part 3 can be rotated around two axis of rotation and be provided with higher flexibility in obtaining an optimal or even different and/or multiple positions for loading of the bag. Hereby, the loading position can also be further away from the wall part 5 compared to a single rotation joint.

FIG. 1e shows schematically a rigid housing le according to another embodiment of the invention. In this embodiment a first joint 11d, a second joint 53 and an extension rod 51 are provided exactly the same as described in relation to FIG. 1d. The only difference in this embodiment is that the bottom part 3e also comprises a door part 61. In this embodiment the door part 61 does not cover the whole height of the tube wall but only a part of the height. Otherwise the embodiment is similar to the embodiment shown in FIG. 1c but provided with two joints, an extension rod and an ability to rotate the bottom part 3e around two axes of rotation.

The extension rod 51 as described in relation to FIGS. 1d and 1e can be designed in different ways. It could be bent as shown or straight. It could also be adjustable such as extensible.

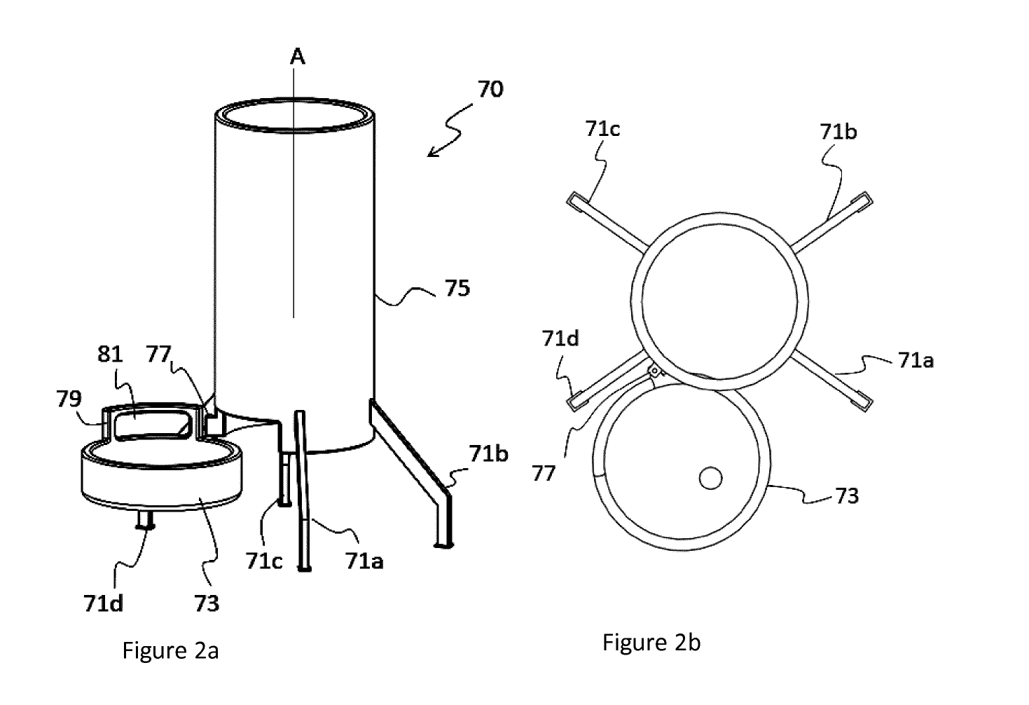

FIG. 2a shows schematically a rigid housing 70 according to one embodiment of the invention where the rigid housing now is provided with legs. In this embodiment four legs 71a, b, c, d are shown connected to a wall part 75 of the rigid housing. However another number of legs could also be provided. The form and position of the legs are adapted for allowing a bottom part 73 of the rigid housing to rotate out from the wall part 75 to a loading position where a flexible bag easily can be provided into the bottom part 73. To allow the rotation the rigid housing needs to be elevated from a floor. This is provided by the legs. The legs 71a, b, c, d also need to be designed and positioned in a way such that the bottom part 73 has enough space to rotate out from the wall part 75. The bottom part 73 is in this embodiment connected to the wall part 75 through a joint 77 such that the bottom part 73 can be rotated out from the wall part about an axis of rotation that is substantially parallel with a longitudinal axis A of the rigid housing as described above. In another embodiment the bottom part could instead be connected to one of the legs or to another part of a stand provided to the wall part of the rigid housing. The connection would also in that embodiment be through a joint such that the bottom part can be rotated to a processing position right below the wall part and to a loading position separated from the wall part. In the embodiment shown in FIG. 2a the bottom part 73 comprises a small door part 79 with an opening 81. Correspondingly the wall part 75 comprises a small opening 83 which will be closed by the small door part 79 of the bottom part 73 when the bottom part 73 is provided beneath the wall part 75, i.e. when it is not in loading position. This small door part 79 is provided for supporting the flexible bag at its connection points. Hereby the flexible bag can be provided with its ports in a correct position already during loading of the flexible bag into the bottom part.

FIG. 2b shows schematically the rigid housing 70 of FIG. 2a in a top view. Here the joint 77 between the bottom part 73 and the wall part 75 can be seen.

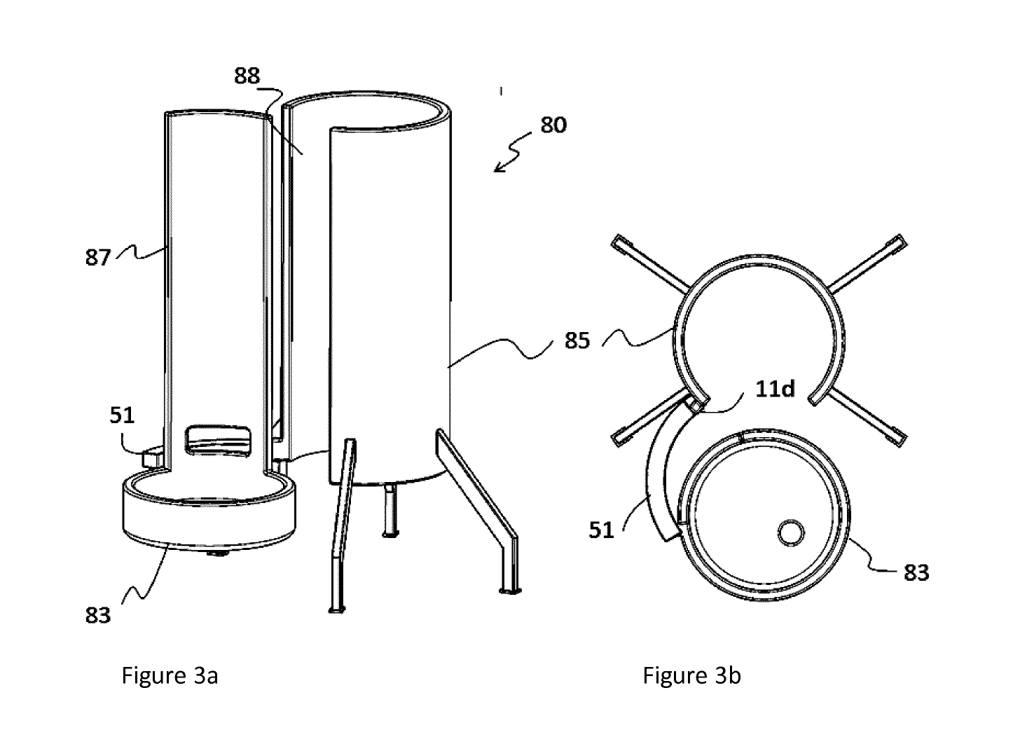

FIG. 3a shows schematically a rigid housing 80 according to one embodiment of the invention. Also in this embodiment four legs 71a, 71b, 71c, 71d are provided to the wall part 85. In this embodiment an extension rod 51 is provided as described in relation to FIG. 1d and le. A first joint 11d is provided as connection between one end of the extension rod 51 and the wall part 85 and a second joint 53 is provided as connection between the bottom part 83 and the other end of the extension rod 51. These first and second joints 11d, 53 can be seen in FIG. 3b which is a top view of the rigid housing 80 shown in FIG. 3a. The bottom part 83 comprises a door part 87 which covers an opening 88 over the whole height of the wall part when the bottom part 83 is rotated in below the wall part, i.e. when the bottom part is not in a loading position, as previously described in relation to FIG. 1c.

In one embodiment of the invention the bottom part of the rigid housing comprises at least one fluid conduit or electrical cable connected to a supply and/or control system, which fluid conduit or electrical cable is routed between the supply and/or control system and the bottom part such that there will be substantially no axial displacement along a direction of said conduit or cable when the bottom part is moved between the loading position and the processing position.

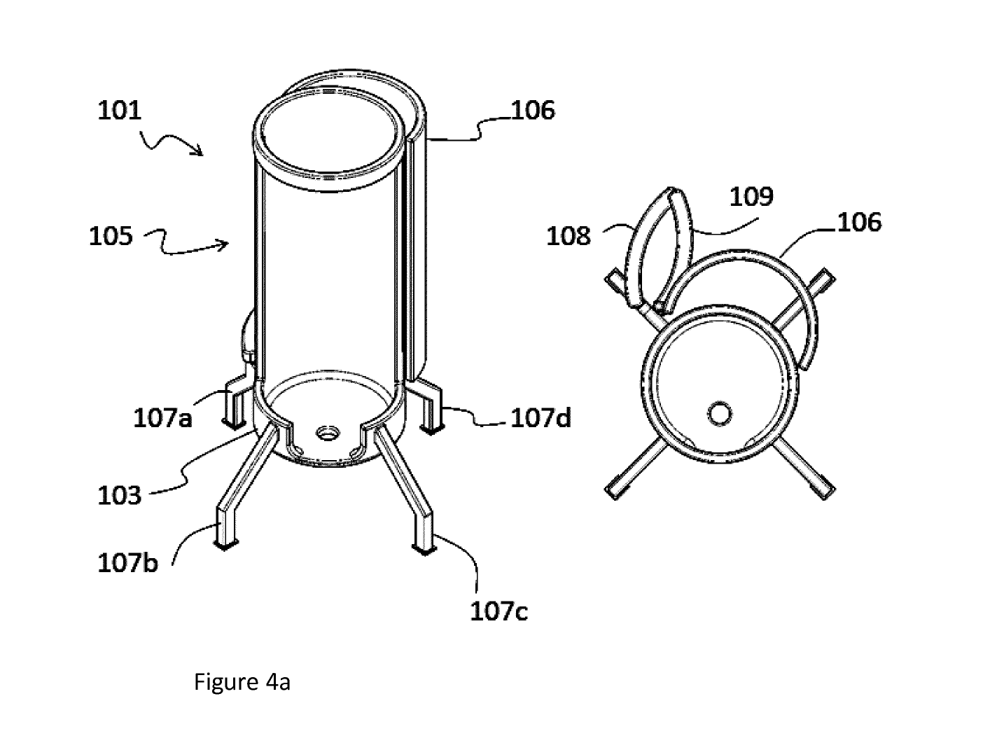

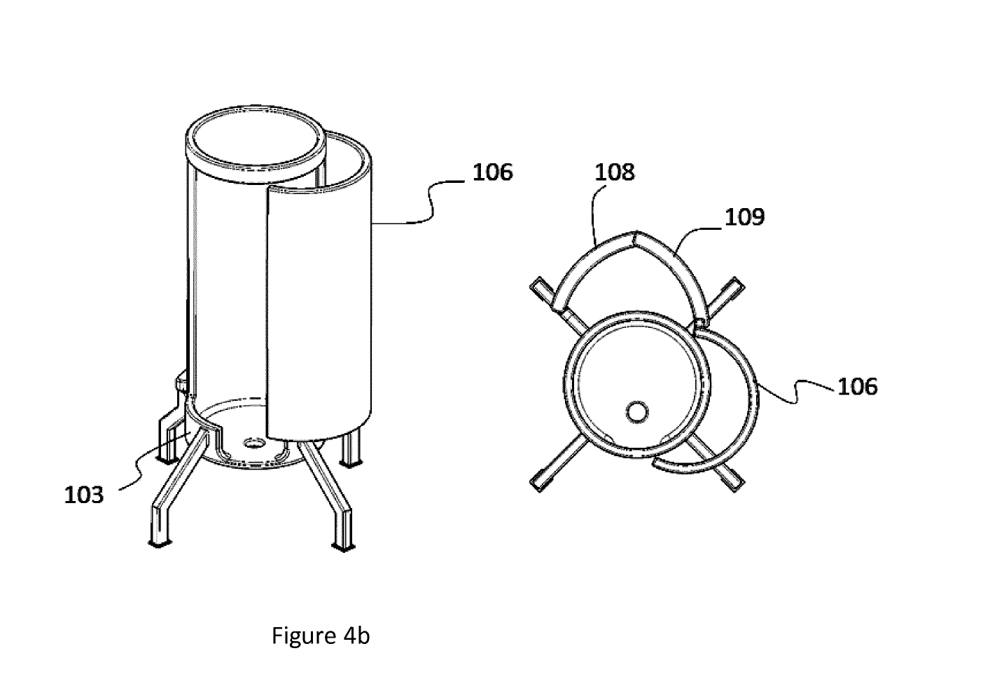

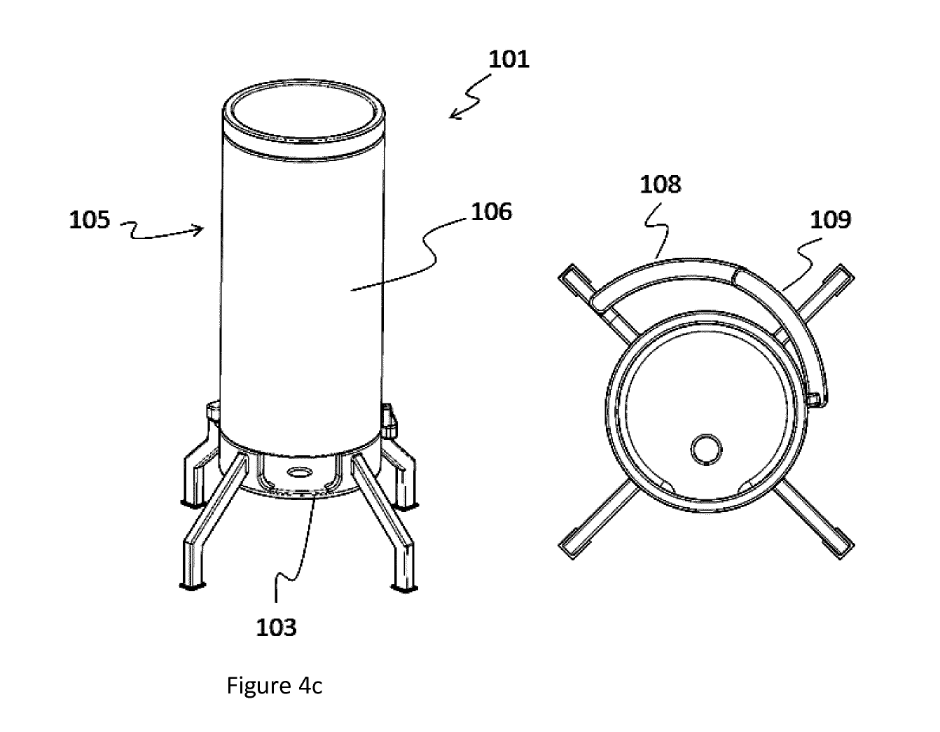

FIG. 4a shows schematically a rigid housing 101 according to another embodiment of the invention. In this embodiment of the invention a bottom part 103 of the rigid housing 101 is fixed, i.e. cannot be rotated as in the previous embodiments. According to the invention access is needed to the bottom part 103 for loading a flexible bag into it. In this embodiment access is achieved by opening and rotating a part of a side wall 105 of the rigid housing 101. That part of the side wall 105 is here called a front part 106. A multiple joint configuration is provided for allowing the front part 106 of the side wall 105 to be opened and then folded up behind the rigid housing. Hereby space is saved in the room. The front part 106 is a part of the side wall big enough for giving good access to the bottom part 103 when the front part is in an open position, also called a loading position. The front part 106 can be extending over the whole height of the side wall as shown in FIGS. 4a-4c but it can also be a part of the height of the side wall. The front part 106 is also extending over a part of the circumference of the side wall, Legs are provided to the rigid housing for lifting it from the floor. Here four legs 107a,b,c,d are shown attached to the bottom part. The multiple joint configuration is here embodied as a first bar 108 pivotally attached to one of the legs and a second bar 109 pivotally attached to the first bar 108 and to the front part 106. Hereby there are three axis of rotation and they are all substantially parallel to a longitudinal axis of the rigid housing. In FIG. 4a the front part 106 is provided in a fully open position, called a first position or a loading position. The front part 106 is folded up behind the rest of the side wall. The first and second bars have been pivoted as is shown in the top view of FIG. 4a such that the front part can be positioned behind the rest of the side wall. If the front part is big and heavy additional pivoting bars may be needed to be provided at other heights of the front part.

FIG. 4b shows the embodiment of FIG. 4a but in a second position. In the second position the front part has been opened but not yet folded up behind the rigid housing 101.

FIG. 4c shows the embodiment of FIG. 4a in a third position also called a processing position. In the third position the front part 106 is closed and the rigid housing is ready for operation.

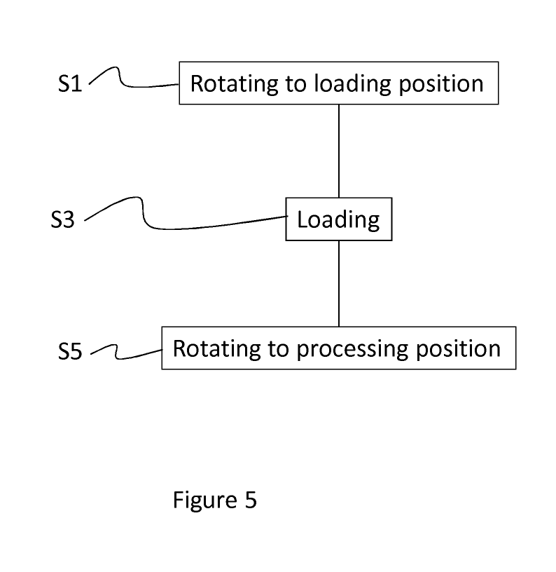

FIG. 5 is a flow chart of a method for providing a flexible bag into a rigid housing as described above. The steps of the method are described below.

S1: Rotating either a bottom part 3, 3c, 3d, 3f, 73, 83 and/or a wall part 106 of the rigid housing 1a, 1b, 1c, 1d, 1e, 1f, 70, 80, 101 to a loading position. The rotation is about an axis of rotation which is substantially parallel with a longitudinal axis A of the rigid housing.

S3: Loading a flexible bag into the bottom part 3, 3c, 3d, 3f, 73, 83. The flexible bag is provided into the bottom part which suitably is container formed to keep the flexible bag inside it.

S5: Rotating either the bottom part and/or the wall part back to a processing position where the bottom part and the wall part forms a rigid housing with an internal volume.

In one embodiment of the invention the method further comprises the step of opening a door in the at least one wall part for allowing an impeller to be provided together with the flexible bag to the internal volume of the rigid housing.

In one embodiment of the invention the method further comprises the step of connecting for example cables, sensors, tubes and/or mixing device connections to ports or access points at the flexible bag. This step of establishing connections or parts of such steps for establishing connections could be performed prior to step 5.

The invention also discloses a bioreactor comprising a flexible bag mounted in a rigid housing as described above. The flexible bag can suitably be loaded in the rigid housing according to the methods described above. Suitably, the flexible bag may contain a magnetic impeller to provide agitation. The bag may further comprise a sparger for gas addition.

Further, the invention discloses use of the bioreactor for the cultivation of cells in the flexible bag of the bioreactor, as well as a method of cultivating cells in the flexible bag of the bioreactor, comprising the steps of providing the bioreactor with the flexible bag loaded in the rigid housing, adding culture medium and cells to the bag and cultivating cells under agitation.

1. A rigid housing comprising a bottom part and at least one wall part, said bottom part and said at least one wall part together defining an internal volume when the bottom part is provided below the at least one wall part in a processing position, said rigid housing being arranged for holding a flexible bag within the internal volume, wherein the bottom part is rotatable about an axis of rotation, wherein said axis of rotation is substantially parallel to a longitudinal axis of said rigid housing such that the bottom part can be provided in a loading position in which the bottom part has been rotated out from the position below the at least one wall part.

2. A rigid housing according to claim 1, wherein the flexible bag is a single use bioreactor.

3. A rigid housing according to claim 1, further comprising a joint connected to the bottom part and to the at least one wall part or to a stand connected to the at least one wall part, said joint providing the axis of rotation about which the bottom part is rotatable.

4. A rigid housing according to claim 1, wherein the at least one wall part comprises at least one opening that allows for the transfer of liquid, access to measuring of parameters or properties of the fluid internal to the bioreactor and/or coupling a mixer element internal to the bag to an external drive unit.

5. A rigid housing according to claim 1, wherein the bottom part comprises a door part which is closing at least one opening of the wall part when the bottom part is provided below the wall part in the processing position.

6. A rigid housing according to claim 1, wherein the bottom part comprises a bottom plate and surrounding walls extending from the bottom plate forming a container together with the bottom plate.

7. A rigid housing according to claim 1, wherein the bottom part comprises one or more openings which allow for the transfer of liquid, access to measuring of parameters or properties of the fluid internal to the flexible bag and/or coupling a mixer element provided in the flexible bag to an external drive unit.

8. A rigid housing according to claim 1, further comprising an extension rod connected by a first joint to the at least one wall part or to a stand connected to the at least one wall part and by a second joint to the bottom part hereby providing two axis of rotation both being substantially parallel to a longitudinal axis of said rigid housing.

9. A rigid housing according to claim 1, wherein the bottom part comprises at least one fluid conduit or electrical cable connected to a supply and/or control system, which fluid conduit or electrical cable is routed between the supply and/or control system and the bottom part such that there will be substantially no axial displacement along a direction of said conduit or cable when the bottom part is moved between the loading position and the processing position.

10. A method for providing a flexible bag into a rigid housing according to claim 1, said method comprising the steps of:
rotating the bottom part of the rigid housing to a loading position;
loading the flexible bag into the bottom part;
rotating the bottom part back to a processing position where the bottom part and the at least one wall part forms a rigid housing with an internal volume.

11. A method according to claim 10, further comprising the step of opening a door in the at least one wall part for allowing an impeller to be provided together with the flexible bag to the internal volume of the rigid housing.

12. A method according to claim 10, further comprising the step of connecting cables and/or sensors and/or tubes to ports provided in the flexible bag.

13. A rigid housing comprising a bottom and at least one wall part comprising a front part, said bottom part and said at least one wall part together defining an internal volume when the front part is provided in a processing position, said rigid housing being arranged for holding a flexible bag within the internal volume, wherein the front part is attached to a multiple joint configuration which also is attached to another part of the rigid housing such that the front part can be provided both in a closed position, also called a processing position, where the front part together with the rest of the side wall enclose the internal volume and in a folded up position, also called a loading position, where the front part is folded up behind the rest of the side wall and access is given to the bottom part for loading of a flexible bag into the bottom part.

14. A bioreactor comprising a flexible bag mounted in a rigid housing according to claim 1.

15. The bioreactor according to claim 14, wherein said flexible bag has been loaded into a rigid housing, comprising the steps of:
rotating the bottom part of the rigid housing to a loading position;
loading the flexible bag into the bottom part;
rotating the bottom part back to a processing position where the bottom part and the at least one wall part forms a rigid housing with an internal volume.

16. Use of the bioreactor according to claim 14 for the cultivation of cells in said flexible bag.

17. A method of cultivating cells in the flexible bag of claim 14, comprising the steps of providing the bioreactor with the flexible bag loaded in the rigid housing, adding culture medium and cells to the bag and cultivating cells under agitation.
